(19) United States
(12) Patent Application Publication
(10) Pub. No.: US 2007/0238772 A1
(43) Pub. Date: Oct. 11, 2007

(54) Use of Metronidazole for Preparing a Pharmaceutical Composition for Treating a Cutaneous Vascularisation Disorder

(75) Inventors: Fabrizio Dolfi, Valbonne (FR); Irina Safonova, Nice (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: Galderma Research & Development, S.N.C., Valbonne Sophia Antipolis (FR)

(21) Appl. No.: 10/590,074

(22) PCT Filed: Feb. 17, 2005
(86) PCT No.: PCT/FR05/00369

§ 371 Date: Apr. 10, 2007

(30) Foreign Application Priority Data
Feb. 20, 2004 (FR) 04/01720

(51) Int. Cl. A61K 31/4164; A61P 17/00

(52) U.S. Cl. 514/398

(57) ABSTRACT

The invention relates more particularly to the use of metronidazole for the preparation of a pharmaceutical composition for treating a cutaneous vascularization disorder.

The present invention relates to the field of cutaneous vascularization disorders, and more particularly to the treatment of cutaneous vascularization disorders in rosacea. The invention is directed towards providing novel pharmaceutical compositions, more particularly dermatological compositions, which are useful for treating cutaneous vascularization disorders, and more particularly for treating cutaneous vascularization disorders in rosacea, and comprising metronidazole as active agent.

Rosacea is a common, chronic and progressive inflammatory dermatitis associated with vascular instability. It mainly affects the central part of the face and is characterized by redness of the face or hot flushes, facial erythema, papules, pustules and telangiectasia. In serious cases, especially in men, the soft tissue of the nose may swell and produce a bulbous swelling known as rhinophyma.

Rosacea generally occurs between the ages of 25 and 70, and is much more common in people of fair complexion. It more particularly affects women, although this affection is generally more severe in men. Rosacea is chronic and lasts for years with periods of exacerbation and of remission.

Rosacea was originally called “acne rosacea” because its papules (points of slight raising of the skin) and its inflammatory pustules (pus scabs) greatly resemble those of common acne. In contrast with common acne, whose aetiology is based on abnormal keratinization, an increase in sebum production and also bacterial inflammation, the inflammation of rosacea is vascular in nature and is poorly understood. The result of this facial vascular anomaly is a permanent oedema of the dermis, which may be accompanied by an increased colonization with Demodex folliculorum, a mite usually found in the follicles of the face.

According to various studies, Demodex folliculorum is thought to have an aetiological role in rosacea (Erbagi et al. 1998, Int. J. Dermatol., vol. 37, pages 421-425; Purcell et al. 1986, J. Am. Acad. Dermatol., vol. 15, pages 1159-1162; Sibenge et al. 1992, J. Am. Acad. Dermatol., vol. 26, pages 590-593). It appears that Demodex folliculorum causes or aggravates inflammatory reactions, reflected by papules and pustules, by blocking the pilosebaceous follicles of the face (Roihu et al. 1998, J. Cutan. Pathol., vol. 25, pages 550-552). This parasite is moreover thought to trigger a humoral immune response (Nunzi et al. 1980, Br. J. Dermatol., vol. 103, pages 543-551; Manna et al. 1982, Br. J. Dermatol., vol. 107, pages 203-208).

The pathogenesis of rosacea is poorly understood. Many factors may be involved without necessarily inducing this complaint. They are, for example, psychological factors, gastrointestinal disorders, environmental factors (exposure to sunlight, temperature, humidity), emotional factors (stress), dietary factors (alcohol, spices), hormonal factors or vascular factors, or even infection with Helicobacter pilori.

Rosacea develops in four stages, but passage through all the stages is not obligatory:

stage 1 of vascular relaxation (at about 20 years old). The patients have sudden bursts of paroxystic redness of the face and neck, with a hot sensation, but with no systemic signs. After the attacks, the skin of the face returns to normal. These “flushes” are triggered by changes in temperature (occasionally leading to thermophobia), and the intake of hot drinks or alcohol;

stage 2 of erythemato-telangiectasia (at about 30 years old). The cheekbone areas are diffusely red. Dilated capillaries constituting standard acne rosacea are observed therein. In contrast with stage 1, the redness is permanent. Besides the cheeks, the chin and the middle of the forehead may be affected;

stage 3 of papulo-pustules (at about 40 years old). Papules and pustules a few millimetres in diameter develop on a background of erythema, without associated comedones. This dermatitis may be very extensive, occasionally up to the bald part of the scalp in men, but is absent from the area around the mouth and the eyes. The patients complain of sensitive skin, with subjective intolerance to the majority of topical products and greasy cosmetics;

stage 4 of rhinophyma (at about 50 years old or later). This late phase mainly affects men, in contrast with the other stages. The nose is increased in volume and diffusely red, and the follicular orifices are dilated. The skin gradually thickens.

The minor forms of rosacea may be treated with active agents such as anti-seborrhoeic agents and anti-infectious agents, for example benzoyl peroxide and retinoic acid. As regards the most diffuse forms of the complaint, they respond well to general antibiotic therapy with cyclines. However, these treatments have unpleasant side effects for the patient, such as irritation or intolerance phenomena.

Furthermore, on account of the multi-factor aspect of rosacea, there are a huge number of treatments for this condition, but the search continues for an effective treatment that is without risk for the patient.

The Applicant's studies have demonstrated the interaction of metronidazole with receptors chosen from the group comprising the beta-adrenergic receptors, the AT1 receptor, the 5-HT2 receptor, the 5-HT5 receptor and the galanin receptor in the treatment of rosacea.

The beta-adrenergic receptors are involved in regulating various physiological functions, such as metabolic activity, cardiac activity, respiration, central nervous system activity, the blood pressure and the vascular tonus.

The 5-HT2 receptors and the 5-HT5 receptors belong to the family of serotonin (5-HT) receptors. The 5-HT receptors are all coupled to G proteins, except for 5-HT3, which is an ion channel. Activation of the 5-HT2 receptors stimulates the activity of phospholipase C. The 5-HT5 receptor transduction system is positively associated with adenylate cyclase.

The AT1 receptor is involved in regulating vasoconstriction by angiotensin II. In man, angiotensin II increases the tonus of the subcutaneous arteries.

Galanin is a 29-amino-acid peptide present in the central nervous system. According to certain studies, galanin is thought to play a role in modulating the cutaneous vascular reaction and in inflammation (Pincelli, 1990, Br. J. Dermatol., vol. 122, pages 745-750).

Metronidazole, or (2-methyl-5-nitroimidazolyl)-2-ethanol, is known in the prior art for its anti-bacterial, anti-parasitic and anti-protozoan properties. It exerts selective toxicity on anaerobic microorganisms and also on hypoxic cells. In the latter, metronidazole is reduced to derivatives capable of impairing the DNA structure of these cells.

The Applicant's studies have demonstrated the involvement of the beta-adrenergic receptors, the AT1 receptor, the 5-HT2 receptor, the 5-HT5 receptor and the galanin receptor in cutaneous vascularization disorders.

The Applicant has now demonstrated the advantageous properties of metronidazole on cutaneous vascularization disorder, and more particularly cutaneous vascularization disorder in rosacea.

It has been found, surprisingly, that the use of metronidazole has as a consequence an interaction with the beta-adrenergic receptors, the AT1 receptor, the 5-HT2 receptor, the 5-HT5 receptor and the galanin receptor. More particularly, it has been found that the use of metronidazole inhibits the binding of the natural ligands to these receptors.

As indicated previously, the invention is directed towards offering a novel method for treating a cutaneous vascularization disorder, which consists in administering to a person suffering from cutaneous vascularization disorder an effective amount of metronidazole, in which the metronidazole is capable of interacting with at least one receptor chosen from the group comprising the beta-adrenergic receptors, the AT1 receptor, the 5-HT2 receptor, the 5-HT5 receptor and the galanin receptor.

Consequently, the invention relates more particularly to the use of metronidazole for the preparation of a pharmaceutical composition for treating a cutaneous vascularization disorder.

More particularly, the invention relates to the use of metronidazole for the preparation of a pharmaceutical composition for treating a cutaneous vascular disorder, involving at least one receptor chosen from the group comprising the beta-adrenergic receptors, the AT1 receptor, the 5-HT2 receptor, the 5-HT5 receptor and the galanin receptor.

The invention also relates to the use of metronidazole for the preparation of a pharmaceutical composition for treating a cutaneous vascularization disorder, involving at least two receptors chosen from the group comprising the beta-adrenergic receptors, the AT1 receptor, the 5-HT2 receptor, the 5-HT5 receptor and the galanin receptor.

The invention also relates to the use of metronidazole for the preparation of a pharmaceutical composition for treating a cutaneous vascular disorder, involving at least three receptors chosen from the group comprising the beta-adrenergic receptors, the AT1 receptor, the 5-HT2 receptor, the 5-HT5 receptor and the galanin receptor.

The invention also relates to the use of metronidazole for the preparation of a pharmaceutical composition for treating a cutaneous vascular disorder, involving at least four receptors chosen from the group comprising the beta-adrenergic receptors, the AT1 receptor, the 5-HT2 receptor, the 5-HT5 receptor and the galanin receptor.

The invention also relates to the use of metronidazole for the preparation of a pharmaceutical composition for treating a cutaneous vascular disorder, involving at least five receptors chosen from the group comprising the beta-adrenergic receptors, the AT1 receptor, the 5-HT2 receptor, the 5-HT5 receptor and the galanin receptor.

More particularly, the invention relates to the use of metronidazole for the preparation of a pharmaceutical composition for treating a cutaneous vascular disorder, the said vascular disorder being a component of rosacea and the metronidazole of the said composition being capable of interacting with at least one receptor chosen from the group comprising the beta-adrenergic receptors, the AT1 receptor, the 5-HT2 receptor, the 5-HT5 receptor and the galanin receptor.

The invention also relates to the use of metronidazole for the preparation of a pharmaceutical composition as defined above, in which the metronidazole is capable of interacting with at least two receptors chosen from the group comprising the beta-adrenergic receptors, the AT1 receptor, the 5-HT2 receptor, the 5-HT5 receptor and the galanin receptor.

The invention also relates to the use of metronidazole for the preparation of a pharmaceutical composition as defined above, in which the metronidazole is capable of interacting with at least three receptors chosen from the group comprising the beta-adrenergic receptors, the AT1 receptor, the 5-HT2 receptor, the 5-HT5 receptor and the galanin receptor.

The invention also relates to the use of metronidazole for the preparation of a pharmaceutical composition as defined above, in which the metronidazole is capable of interacting with at least four receptors chosen from the group comprising the beta-adrenergic receptors, the AT1 receptor, the 5-HT2 receptor, the 5-HT5 receptor and the galanin receptor.

The invention also relates to the use of metronidazole for the preparation of a pharmaceutical composition as defined above, in which the metronidazole is capable of interacting with at least five receptors chosen from the group comprising the beta-adrenergic receptors, the AT1 receptor, the 5-HT2 receptor, the 5-HT5 receptor and the galanin receptor.

More particularly, the invention relates to the use of metronidazole for the preparation of a pharmaceutical composition in which the metronidazole inhibits the binding of at least one natural ligand to its receptor, the said receptor being chosen from the group comprising the beta-adrenergic receptors, the AT1 receptor, the 5-HT2 receptor, the 5-HT5 receptor and the galanin receptor.

The composition that is the subject of the present invention is a dermatological composition for topical administration to the skin.

According to the present invention, the term “treatment of cutaneous vascularization disorder” means the treatment and/or prevention of such a disorder.

According to the present invention, the term “treatment of rosacea” means the treatment and/or prevention of rosacea, at one or more of the stages described above.

According to a first embodiment of the invention, the composition is intended for treating the first stage of rosacea.

According to a second embodiment of the invention, the composition is intended for treating the second stage of rosacea.

According to a third embodiment of the invention, the composition is intended for treating the third stage of rosacea.

According to a fourth embodiment of the invention, the composition is intended for treating the fourth stage of rosacea.

According to one preferential embodiment, the composition contains from 0.0001% to 20% by weight of metronidazole, preferably from 0.1% to 2% and more preferentially from about 0.75% to 1% of metronidazole expressed as weight percentages relative to the total weight of the composition.

Needless to say, the present invention concerns, besides the use of metronidazole, the use of derivatives thereof. The term “derivatives” means compounds that differ from metronidazole by substitution, addition or removal of one or more chemical groups and that have substantially the same activity.

Advantageously, the compositions of the invention comprise, besides metronidazole, at least one other therapeutic agent capable of increasing the efficacy of the treatment. Non-limiting examples of such agents that may be mentioned include antibiotics, antibacterial agents, antiviral agents, antiparasitic agents, antifungal agents, anaesthetics, analgesics, antiallergic agents, retinoids, free-radical scavengers, anti-pruriginous agents, keratolytic agents, anti-seborrhoeic agents, antihistamines, sulfides, immunosuppressant products and anti-proliferative products.

The compositions of the invention may also comprise any additive usually used in the pharmaceutical or dermatological field that is compatible with metronidazole. Mention may be made especially of sequestrants, antioxidants, sunscreens, preserving agents, for example DL-α-tocopherol, fillers, electrolytes, humectants, dyes, common mineral or organic acids or bases, fragrances, essential oils, cosmetic active agents, moisturizers, vitamins, essential fatty acids, sphingolipids, self-tanning compounds such as DHA, skin calmative and protective agents such as allantoin, pro-penetrating agents and gelling agents. Needless to say, a person skilled in the art will take care to select this or these optional additional compound(s), and/or the amount thereof, such that the advantageous properties of the composition according to the invention are not, or are not substantially, adversely affected.

These additives may be present in the composition in a proportion of from 0 to 20% by weight relative to the total weight of the composition.

Examples of sequestrants that may be mentioned include ethylenediaminetetraacetic acid (EDTA), and also derivatives or salts thereof, dihydroxyethylglycine, citric acid and tartaric acid, or mixtures thereof.

Examples of preserving agents that may be mentioned include benzalkonium chloride, phenoxyethanol, benzyl alcohol, diazolidinylurea and parabens, or mixtures thereof.

Examples of humectants that may be mentioned include glycerol and sorbitol.

The compositions of the invention may contain one or more pro-penetrating agents in preferential concentrations ranging from 0 to 20% and more preferentially ranging from 0.6% to 3% by weight relative to the total weight of the composition. Among the pro-penetrating agents that are preferentially used, without this list being limiting, are compounds such as propylene glycol, dipropylene glycol, propylene glycol dipelargonate, lauroglycol and ethoxydiglycol.

Advantageously, the compositions according to the invention may also contain one or more wetting liquid surfactants in preferential concentrations ranging from 0 to 10% and more preferentially ranging from 0.1% to 2%.

The compositions of the present invention may be in any galenical form normally used for topical application, especially in the form of aqueous, aqueous-alcoholic or oily solutions, dispersions of the lotion type, aqueous, anhydrous or lipophilic gels, emulsions of liquid or semi-liquid consistency of the milk type, obtained by dispersing a fatty phase in an aqueous phase (O/W) or, conversely, (W/O), or suspensions or emulsions of soft, semi-solid or solid consistency of the cream, gel or ointment type, or alternatively microemulsions, microcapsules, microparticles or vesicular dispersions of ionic and/or nonionic type.

Preferably, the creams may be formulated from a mixture of mineral oil or from a mixture of beeswax and of water, which emulsifies instantaneously, to which is added metronidazole, dissolved in a small amount of oil such as almond oil.

The ointments may be formulated by mixing a solution of metronidazole in an oil such as almond oil in warmed paraffin, followed by leaving the mixture to cool.

As examples of compositions according to the invention, mention may be made of those comprising an active phase containing (expressed as weight percentages):

0 to 90%, preferentially 5% to 25% and especially 10% to 20% of water;

0 to 10%, preferentially 0 to 2% and especially 0 to 0.5% of wetting liquid surfactant;

0 to 20%, preferentially 0 to 10% and especially 2% to 5% of pro-penetrating agent;

0.0001% to 20% and preferentially 0.1% to 2% of metronidazole;

and an aqueous phase comprising a pH-independent gelling agent, and water.

The aqueous phase of a composition according to the invention in the form of an emulsion may comprise water, a floral water such as cornflower water or a natural spring or mineral water chosen, for example, from eau de Vittel, the waters of the Vichy basin, eau d'Uriage, eau de la Roche Posay, eau de la Bourboule, eau d'Enghien-les-Bains, eau de Saint Gervais-les-Bains, eau de Néris-les-Bains, eau d'Allevard-les-Bains, eau de Digne, eau de Maiziéres, eau de Neyrac-les-Bains, eau de Lons-le-Saunier, les Eaux Bonnes, eau de Rochefort, eau de Saint Christau, eau des Fumades, eau de Tercis-les-Bains, eau d'Avéne and eau d'Aix-les-Bains.

The said aqueous phase may be present in a content of between 10% and 90% by weight and preferably between 20% and 80% by weight relative to the total weight of the composition.

Non-limiting examples that may be mentioned include gelling agents of the polyacrylamide family such as the sodium acryloyldimethyltaurate copolymer/isohexa-decane/polysorbate-80 mixture sold under the name Simulgel 600 by the company SEPPIC, the polyacrylamide/C13-14 isoparaffin/laureth-7 mixture, for instance the product sold under the name Sepigel 305 by the company SEPPIC, the family of acrylic polymers coupled to hydrophobic chains, such as the PEG-150/decyl/SMDI copolymer sold under the name Aculyn 44 (polycondensate comprising at least, as components, a polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%)), and the family of modified starches such as the modified potato starch sold under the name Structure Solanace, or mixtures thereof.

The preferred gelling agents are derived from the polyacrylamide family, such as Simulgel 600 or Sepigel 305 or mixtures thereof.

The gelling agent as described above may be used in preferential concentrations ranging from 0.1% to 15% and more preferentially ranging from 0.5% to 5%.

The gels may preferably be prepared by dispersing or dissolving metronidazole in a suitable ratio in a gel of carbomer, poloxamer or cellulose-based type.

Other advantages and characteristics of the invention will emerge from the examples below concerning the activity of metronidazole.

### EXAMPLE 1

### Activity of Metronidazole

1) Protocol:

The test of binding to the beta-1 and beta-2 adrenergic receptors was performed according to the method described by Smith and Teiler 1999, Cardiovasc. Drug Ther., vol. 13, pages 123-126.

The test of binding to the AT1 receptor was performed according to the method described by Bergsma et al., 1992, Biochem. Biophys. Res. Comm., vol. 183, pages 989-995.

The test of binding to the 5-HT_{5A} receptor was performed according to the method described by Ress et al., 1994, FEBS Lett., vol. 355, pages 242-246.

The test of binding to the 5-HT_{2A} receptor was performed according to the method described by Bonhauss et al., 1995, Brit. J. Pharmacol., vol. 1155, pages 622-628.

The test of binding to the galanin receptor was performed according to the method described by Sullivan et al., 1997, Biochem. Biophys. Res. Comm., vol. 233, pages 823-828.

2) Experimental Conditions:

The binding of metronidazole to each receptor was determined by competitive experiments. The receptor, human recombinant protein, was incubated for times indicated in Table 1 below, with a simple concentration of labelled specific ligand, in the presence of 10 μM metronidazole. The bound radioactivity was measured by scintillation counting.

TABLE 1

| | Radiolabelled | | Incubation |
|---|---|---|---|
| Receptor | specific ligand | Non-specific ligand | conditions |
| Beta₁ | [³H]CGP 12177 | Aprenolol (50 μM) | 60 min/22° C. |
| adrenergic | (0.15 nM) | | |
| Beta₂ | [³H]CGP 12177 | Aprenolol (50 μM) | 60 min/22° C. |
| adrenergic | (0.15 nM) | | |
| AT1 | [¹²⁵I][Sar¹Ile⁸] | Angiotensin II | 60 min/22° C. |
| | AII (0.05 nM) | (10 μM) | |
| 5-HT2A | [³H]ketanserin | Ketanserin (1 μM) | 15 min/37° C. |
| | (0.5 nM) | | |
| 5-HT5A | [³H]LSD (1 nM) | Serotonin (100 μM) | 30 min/37° C. |
| Galanin 1 | [¹²⁵I]galanin | Galanin (1 μM) | 60 min/22° C. |
| | (0.03 nM) | | |

3) Analysis and Expression of the Results:

The specific binding of the ligand to the receptor is defined as the difference between the total binding and the non-specific binding determined in the presence of an excess of unlabelled ligand.

The results are expressed as a percentage of control specific binding and as a percentage of inhibition of the control specific binding obtained in the presence of metronidazole (Table 2).

TABLE 2

| | | % of control specific |
|---|---|---|
| Receptor | Metronidazole (μM) | binding (±SD) |
| Beta₁ adrenergic | 10 | 85.1 ± 1.9 |
| Beta₂ adrenergic | 10 | 85.9 ± 4.3 |
| AT1 | 10 | 79.7 ± 2.0 |
| 5-HT2A | 10 | 81.6 ± 0.8 |
| 5-HT5A | 10 | 83.2 ± 3.1 |
| Galanin 1 | 10 | 81.1 ± 3.4 |

Metronidazole interacts and thus inhibits the binding to the beta-adrenergic receptors, to the AT1 receptor, to the 5-HT2 receptor, to the 5-HT5 receptor and to the galanin receptor.

1. A pharmaceutical composition for treating a cutaneous vascularization disorder, comprising a cutaneous vascularization disorder treating effective amount of metronidazole.

2. The composition according to claim 1, wherein said cutaneous vascular disorder involves at least one receptor selected from the group consisting of the beta-adrenergic receptors, the AT1 receptor, the 5-HT2 receptor, the 5-HT5 receptor and the galanin receptor.

3. The composition according to claim 1, wherein said cutaneous vascular disorder involves at least two receptors selected from the group consisting of the beta-adrenergic receptors, the AT1 receptor, the 5-HT2 receptor, the 5-HT5 receptor and the galanin receptor.

4. The composition according to claim 1, wherein said cutaneous vascular disorder involves at least three receptors selected from the group consisting of the beta-adrenergic receptors, the AT1 receptor, the 5-HT2 receptor, the 5-HT5 receptor and the galanin receptor.

5. The composition according to claim 1, wherein said cutaneous vascular disorder involves at least four receptors selected from the group consisting of the beta-adrenergic receptors, the AT1 receptor, the 5-HT2 receptor, the 5-HT5 receptor and the galanin receptor.

6. The composition according to claim 1, wherein said cutaneous vascular disorder involves at least five receptors selected from the group consisting of the beta-adrenergic receptors, the AT1 receptor, the 5-HT2 receptor, the 5-HT5 receptor and the galanin receptor.

7. The composition according to claim 1, wherein said vascular disorder is a component of rosacea and metronidazole is capable of interacting with at least one receptor selected from the group consisting of the beta-adrenergic receptors, the AT1 receptor, the 5-HT2 receptor, the 5-HT5 receptor and the galanin receptor.

8. The composition according to claim 1, wherein metronidazole inhibits the binding of at least one natural ligand to its receptor, the receptor being selected from the group consisting of the beta-adrenergic receptors, the AT1 receptor, the 5-HT2 receptor, the 5-HT5 receptor and the galanin receptor.

9. The composition according to claim 1, wherein said pharmaceutical composition is a dermatological composition for topical application.

10. The composition according to claim 1, wherein the composition is for treating at least one stage of rosacea.

11. The composition according to claim 1, wherein the composition is for treating the first stage of rosacea.

12. The composition according to claim 1, wherein the composition is for treating the second stage of rosacea.

13. The composition according to claim 1, wherein the composition is for treating the third stage of rosacea.

14. The composition according to claim 1, wherein the composition is for treating the fourth stage of rosacea.

15. The composition according to claim 1, wherein said composition contains from 0.0001% to 20% by weight, of metronidazole.

16. The composition according to claim 1, further comprising another active agent selected from the group consisting of antibiotics, antibacterial agents, antiviral agents, anti-parasitic agents, antifungal agents, anaesthetics, analgesics, anti-allergic agents, retinoids, free-radical scavengers, anti-pruriginous agents, keratolytic agents, anti-seborrhoeic agents, antihistamines, sulfides, immunosuppressant products and anti-proliferative products.

17. The composition according to claim 1, further comprising an additive selected from the group consisting of sequestrants, antioxidants, sunscreens, preserving agents, fillers, electrolytes, humectants, colorants common mineral or organic acids or bases, fragrances, essential oils, cosmetic active agents, moisturizers, vitamins, essential fatty acids, sphingolipids, self-tanning compounds, skin calmative and protective agents, pro-penetrating agents and gelling agents, or a mixture thereof.

18. The composition according to claim 15, wherein said composition contains from 0.1% to 2% by weight of metronidazole.

19. The composition according to claim 18, wherein said composition contains from about 0.75% to 1% by weight of metronidazole.
